(19) United States
(12) Patent Application Publication
(10) Pub. No.: US 2005/0169839 A1
(43) Pub. Date: Aug. 4, 2005

(54) Biomarker for efficacy of appetite suppressant drugs

(76) Inventors: Tung M. Fong, Somerset, NJ (US); Chun-Pyn Shen, Edison, NJ (US); Leonardus H.T. Van der Ploeg, Rancho Santa Fe, CA (US)

Correspondence Address: MERCK AND CO., INC, P O BOX 2000, RAHWAY, NJ, 07065-0907, US

(21) Appl. No.: 10/506,577

(22) PCT Filed: Mar. 3, 2003
(86) PCT No.: PCT/US03/06437

Related U.S. Application Data
(60) Provisional application No. 60/361,806, filed on Mar. 5, 2002.

(51) Int. Cl. G01N 33/567; A61K 49/00

(52) U.S. Cl. 424/9.2; 435/7.21

(57) ABSTRACT

The present invention relates to a biomarker for the efficacy of appetite suppressant drugs given to humans or other mammals for the treatment of obesity. It further relates to a novel method of determining the efficacy of a test compound given to a subject for the treatment of obesity, wherein the test compound is an appetite suppressant which does not stimulate the release of serotonin. It also relates to a method for following the progress of a therapeutic regime designed to alleviate obesity and to a method for determining the appropriate dosage of an appetite suppressant given to a subject for the treatment of obesity.

### FIELD OF THE INVENTION

The present invention relates generally to the field of appetite suppressant drugs for the treatment of obesity. More specifically, it relates to a biomarker for the efficacy of appetite suppressant drugs given to humans or other mammals for the treatment of obesity.

### BACKGROUND OF THE INVENTION

Obesity is a leading worldwide health concern due to its correlation with cardiovascular disease, non-insulin dependent diabetes mellitus, certain forms of cancer, gallstones, specific respiratory disorders, and an increased overall mortality rate.

Recent advances in molecular research have established that body weight is controlled, in part, by a highly regulated physiological process that maintains a balance between energy intake and energy expenditure. Several of the molecular interactions involved in this process have been identified and serve as targets for the development of obesity therapeutics (for review, see Bray et al., Nature 404: 672-677 (2000)). The most common strategies for the development of anti-obesity pharmaceuticals include the development of drugs that reduce food intake, alter metabolism and/or increase energy expenditure or thermogenesis.

Potential therapeutic drugs that alter food intake are being developed that act by either magnifying signals that suppress food intake or by blocking signals that stimulate food intake. Numerous signaling pathways are involved in the regulation of food intake and provide drug discovery targets, such as the leptin, melanocortin, neuropeptide Y and serotonergic pathways.

One of the many molecules that contribute to the to the regulation of food intake and body weight in rodents is the agouti related protein (AGRP). (Shutter et al., Genes Dev. 11: 593-602 (1997); Hagan et al., Am. J. Physiol. Regul. Integr. Comp. Physiol. 279: R47-R52 (2000)). AGRP likely exerts its effect on these physiological processes through the competitive antagonism of α-MSH, the natural agonist of the melanocortin 3 and 4 receptors (MC3R and MC4R) (Rossi et al., Endocrinology 139: 4428-4431 (1998); Tota et al., Biochemistry 38: 897-904 (1999); and Yang et al., Mol. Endocrinol. 13: 148-155 (1999)). AGRP also interacts with the melanocortin 1 and 5 receptors (MC1R and MC5R) at lower affinity.

AGRP expression has been detected in human, rodent and chicken brain tissue, specifically in the hypothalamus, as well as in several other tissues (Ollmann et al., Science 278: 135-138 (1997); Bicknell et al., J. Neouroendocrinol. 12: 977-982 (2000)). In addition, circulating AGRP was detected in rat and human plasma (Katsuki et al., J. Clin. Endocr. Metab. 86: 1921-1924 (2001); and Li et al., Endocrinology 141: 1942-1950 (2000)).

Katsuki and colleagues have reported an increase in AGRP levels in the plasma of obese men (J. Clin. Endocr. Metab. 86(5): 1921-1924). An upregulation of AGRP mRNA expression in the hypothalamus of wild-type mice was reported after a two day fasting period (Mizuno and Mobbs, Endocrinology 140(2): 814-817 (1999)).

Candidate drugs that target food intake control pathways are eliminated from clinical development if they are associated with undesirable side effects or are not efficacious. Prior art methods of monitoring the efficacy of appetite suppressant drugs include long-term studies of body weight, weight circumference, waist/hip ratio, and body mass index as well as short- and long-term studies monitoring the subjective rating of hunger and food intake of study subjects. Such studies are typically conducted through visual analog scale assessment, questionnaires, and self-reporting after study subjects have taken the candidate drug for several months or more. It would enhance drug development efforts to formulate a method of quickly determining the efficacy of appetite suppressants that is more objective and can be done earlier in the clinical testing process than prior art methods.

### SUMMARY OF THE INVENTION

The present invention relates to a novel method of determining the efficacy of a test compound given to a subject for the treatment of obesity, comprising: (a) assaying a plasma sample from the subject to determine a level of AGRP at a first time point; (b) administering the test compound to the subject; and (c) thereafter assaying a plasma sample from the subject to determine the level of AGRP at a second time point; wherein the test compound is an appetite suppressant which does not stimulate the release of serotonin and wherein an increased level of AGRP at the second time point relative to the first time point is indicative of the efficacy of the test compound in treating obesity.

The present invention also relates to a method for following the progress of a therapeutic regime designed to alleviate obesity, comprising: (a) assaying a plasma sample from a subject to determine a level of AGRP at a first time point; (b) assaying a second plasma sample from the subject to determine a level of AGRP at a second time point, wherein the therapeutic regime is followed by the subject between the first time point and the second time point; and (c) comparing said level at said second time point to the level determined in (a) as a determination of effect of said therapeutic regime.

The present invention further relates to a method for determining the appropriate dosage of an appetite suppressant given to a subject for the treatment of obesity, comprising: (a) assaying a plasma sample from the subject to determine a level of agouti related protein (AGRP) at a first time point; (b) administering the appetite suppressant to the subject; (c) thereafter assaying a plasma sample from the subject to determine the level of AGRP at a second time point, wherein the appetite suppressant does not stimulate the release of serotonin; (d) determining whether the appetite suppressant was administered at the appropriate dosage, wherein a decreased level of AGRP at the second time point relative to the first time point is indicative of the efficacy of the appetite suppressant in treating obesity at the dosage administered; and (e) adjusting dosage as needed.

As used throughout the specification and in the appended claims, the singular forms “a”, “an”, and “the” include plural reference unless the context clearly dictates otherwise.

As used herein, “therapeutic regime” refers to any course of therapy prescribed or recommended by a physician or veterinarian or followed by a subject for the treatment or control of obesity, wherein the course of therapy includes the administration of at least one appetite suppressant. The therapeutic regime may include combination treatment with more than one active pharmaceutical compound or may be the administration of a single appetite suppressant drug. The therapeutic regime may further include other methods of treatment such as diet and exercise, in accordance with a physician or veterinarian recommended treatment plan or a treatment plan proposed by the subject.

As used herein, “appetite suppressant that does not stimulate the release of serotonin” refers to any pharmaceutical compound for use as an appetite suppressant excluding that class of compounds that has a mode of action that primarily includes stimulating the release of serotonin, such as fenfluramine, d-fenfluramine and (±)-3,4-methylene-dimethoxyamphetamine (MDMA). An “appetite suppressant that does not stimulate the release of serotonin” includes appetite suppressant drugs that inhibit the reuptake of serotonin and noradrenalin such as sibutramine. This term is also meant to include compounds that work primarily by a mode of action other than by release of serotonin, but may have the effect of enhancing the release of serotonin caused by serotonin-releasing compounds when given in combination with a serotonin-releasing compound. For example, as defined herein, phentermine is an “appetite suppressant that does not stimulate the release of serotonin” because its primary mode of action is the inhibition of monoamine oxidase (MAO). Although phentermine has been reported to enhance the serotonin release induced by fenfluramine, it has minimal effect on serotonin when given alone (see Wellman and Maher, Int. J. Obesity 23: 723-32 (1999)).

As used herein, “appropriate dosage” refers to the dosage of a known pharmaceutical compound or test compound at which the compound is efficacious in suppressing appetite or inducing satiety. The appropriate dosage may vary with a variety of factors including the species and weight of the subject and the class of compound.

### BRIEF DESCRIPTION OF THE DRAWINGS

FIG. 1 shows the levels of AGRP (pg/mL plasma) in the plasma of human volunteers after an overnight fast (FO) compared to plasma AGRP levels of the same volunteers after a morning meal (mean±S.D., n=17) (see EXAMPLE 1). Panel (A) shows that the average plasma AGRP level decreased by 39% (p=3.1×10⁻⁶, paired-t test) two hours after a meal relative to average plasma AGRP level before the meal. Panel (B) shows the change in plasma AGRP levels (pg/ml) of individual volunteers before the meal (FO) and two hours later.

FIG. 2 shows the level of AGRP (pg/ml) in the plasma of human volunteers after an overnight fast (FO) compared to plasma AGRP levels of the same volunteers after an additional two hours of fasting (mean±S.D., n=15) (see EXAMPLE 1). Panel (A) shows that the average plasma AGRP level increased by 73% (p=0.047, paired t test) after an additional two hours of fasting. Panel (B) shows the initial plasma AGRP levels (pg/mL plasma) of individual volunteers after overnight fasting (FO) and the change in AGRP levels when fasting continued for an additional two hours.

FIG. 3 shows a correlation between body mass index (BMI) and plasma leptin levels (ng/mL) in the study subjects (p=0.0053, n=17). Plasma samples were obtained from the original 17 volunteers at 9 a.m., after an overnight fasting period.

FIG. 4 shows plasma AGRP levels (pg/mL plasma) of 3 month old male DIO rats (see EXAMPLE 2). Group (1) consisted of rats that were fed ad libitum; group (2) consisted of rats that fasted for forty-eight hours, and group (3) consisted of rats that were fed two hours after a forty eight hour fast. Results show a statistically significant difference in plasma AGRP levels between rats in the fed state and rats in the fasted state (n=6 for each group, p=0.01 one-way ANOVA). An unpaired t-test also showed that there was a statistically significant difference between the plasma AGRP level of rats in group 2 and rats in group 3.

FIG. 5 shows the effect of treatment with the appetite suppressant sibutramine on body weight and plasma AGRP levels in male Sprague-Dawley rats (see EXAMPLE 4). Panel A shows the body weight change (g) of rats treated with sibutramine (n=7) compared to rats treated with vehicle alone (n=5). Panel B shows the mean plasma AGRP level (pg/0.1 mL plasma) of each group±S.D.

FIG. 6 shows the effect of treatment with the MC4R agonist Compound A on body weight and plasma AGRP levels in male DIO rats (see EXAMPLE 5). Panel A shows the body weight change (g) of rats treated with Compound A (n=7) compared to rats treated with vehicle alone (n=7). Panel B shows the mean plasma AGRP level (pg/0.1 mL plasma) of each group±S.D.

FIG. 7 shows the effect of treatment with MC4R agonist Compound B on body weight and plasma AGRP levels in male DIO rats (see EXAMPLE 5). Panel A shows the body weight change (g) of rats treated with Compound B (n=6) compared to rats treated with vehicle alone (n=5). Panel B shows the mean plasma AGRP level (pg/0.1 mL plasma) of each group±S.D.

FIG. 8 shows the effect of treatment with S(+) fenfluramine on body weight and plasma AGRP levels in lean rats (see EXAMPLE 6). Panel A shows the body weight change (g) of rats treated with S(+) fenfluramine (n=7) compared to rats treated with vehicle alone (n=5). Panel B shows the mean plasma AGRP level (pg/0.1 mL plasma) of each group±S.D.

FIG. 9 shows the effect of treatment with AM251 on body weight and plasma AGRP levels in lean rats (see EXAMPLE 7). Panel A shows the body weight change (g) of rats treated with AM251 (n=7) compared to rats treated with vehicle alone (n=6). Panel B shows the mean plasma AGRP level (pg/0.1 mL plasma) of each group±S.D.

### DETAILED DESCRIPTION OF THE INVENTION

The agouti related protein (AGRP) was previously shown to contribute to the regulation of food intake and body weight in rodents. Mizuno and colleagues have shown that AGRP mRNA expression is increased in the hypothalamus of wild-type mice after a two day fast (Endocrinology 140(2): 814-817 (1999)). No study of plasma AGRP levels in conjunction with food intake, fasting, or intake of appetite suppressants has been reported in the prior art. Applicants have shown, for the first time, an association between plasma AGRP level and food intake as well as intake of appetite suppressants. The correlation between plasma AGRP levels and weight gain reduction after sibutramine treatment (see EXAMPLE 4) and treatment with MC4R agonist compound A (see EXAMPLE 5) shows that plasma AGRP level may serve as a biomarker of appetite suppressant efficacy. Thus, one aspect of this invention is the use of plasma AGRP level as a biomarker as it is an objective indicator of appetite suppressant efficacy during drug development, allowing the identification of promising candidate drugs to occur earlier in the lengthy drug discovery process.

Ascertaining the efficacy of an appetite suppressant drug using conventional methods can take about three months to complete. Advantageously, using the present invention, the establishment of the efficacy of a pharmaceutical composition to be used for obesity treatment can be made in one week or less, significantly reducing the amount of time necessary to eliminate non-efficacious compounds from drug development. Consequently, the present invention saves resources and funds from being spent on compounds that will eventually be removed from drug development.

Therefore, the present invention relates to a novel method of determining the efficacy of a test compound given to a subject for the treatment of obesity, comprising: (a) assaying a plasma sample from the subject to determine a level of AGRP at a first time point; (b) administering the test compound to the subject; and (c) thereafter assaying a plasma sample from the subject to determine the level of AGRP at a second time point; wherein the test compound is an appetite suppressant which does not stimulate the release of serotonin and wherein a decreased level of AGRP at the second time point relative to the first time point is indicative of the efficacy of the test compound in treating obesity.

The amount of time between the first time point and the second time point defines a treatment test period for the pharmaceutical to be tested. The treatment test period can be from about two hours to about thirty days. Preferably, the treatment test period is at least four hours. Within this test period, the appetite suppressant may be given once daily or in divided doses of more than one time per day. The dosing regimen may also involve once-weekly administration of the appetite suppressant or may be any dosing schedule required for the specific pharmaceutical composition.

It is not necessary for the subject to fast overnight before the commencement of the treatment test period, but the methods of the present invention may be conducted after an overnight or longer fasting period if desired. However, it is preferred that all subjects within a single clinical trial maintain a consistent fasting period or lack thereof.

The novel methods of the present invention may be used during clinical trials to determine promising candidates for obesity therapeutics and to eliminate non-efficacious drugs from development earlier in the drug development process than by using conventional methods. Conventional measurements of appetite suppressant efficacy such as visual analog scale assessment, questionnaires, or self-reporting may be used in conjunction with the present invention to supplement data generated through use of the AGRP biomarker or the methods of the present invention may be used alone.

Pharmaceutical compounds already known to function as appetite suppressants for the treatment of obesity and those in clinical development must be administered to the subject at an appropriate dosage to be efficacious. The appropriate dosage of appetite suppressant compounds is selected in accordance with a variety of factors including type, species, age, weight, sex and medical condition of the patient; the severity of the condition to be treated; the route of administration; the renal, hepatic and cardiovascular function of the patient; and the particular compound thereof employed. A physician or veterinarian of ordinary skill can determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

The present invention has the objective of providing a novel, quantifiable method for determining the appropriate dosage of an appetite suppressant drug that is more reliable than prior art methods. To this end, the present invention relates to a method for determining the appropriate dosage of an appetite suppressant given to a subject for the treatment of obesity, comprising: (a) assaying a plasma sample from the subject to determine a level of agouti related protein (AGRP) at a first time point; (b) administering the appetite suppressant to the subject; (c) thereafter assaying a plasma sample from the subject to determine the level of AGRP at a second time point, wherein the appetite suppressant does not stimulate the release of serotonin; (d) determining whether the appetite suppressant was administered at the appropriate dosage, wherein a decreased level of AGRP at the second time point relative to the first time point is indicative of the efficacy of the appetite suppressant in treating obesity at the dosage administered; and (e) adjusting dosage as needed.

Circulating AGRP was previously shown to be present in the plasma of rodents as well as humans. Therefore, the methods of the present invention may be performed using plasma samples from a human subject or any other animal subject in which circulating AGRP can be detected in plasma. To this end, the present invention may be utilized during clinical trials utilizing animal or human subjects to objectively determine the efficacy of an appetite suppressant given to the subject for the treatment of obesity.

In a preferred embodiment of the present invention, the subject is a human.

In an alternative embodiment of the present invention, the subject is a rodent. In a further embodiment, the subject is a rat.

One skilled in the art will recognize that plasma levels of AGRP in clinical and test samples can be measured by any of several serological or immunological techniques known in the art. Such techniques include, but are not limited to, enzyme-linked immunosorbent antibody (ELISA), radioimmunoassay (RIA), and radioligand binding techniques.

In a preferred embodiment of the present invention, AGRP level in the plasma of the subject is determined by radioimmunoassay (RIA). The RIA method is a sensitive technique that employs isotopically labeled molecules to determine concentration by measuring radioactivity instead of determining concentration through chemical analysis.

In an alternative embodiment of the present invention, plasma AGRP level is determined using the ELISA technique.

In a further embodiment of the present invention, plasma AGRP level is determined using a radioligand binding assay.

In yet another embodiment of the present invention, plasma AGRP level is measured using liquid chromatography.

Pharmaceutical compositions potentially useful as appetite suppressants to be screened by the methods of the present invention may be selected from a class of compounds representing a known mode of action for inhibiting food intake or may be identified based on a novel mode of action not yet described. Numerous classes of compounds representing distinct modes of action have been described which serve as targets for pharmaceutical development of obesity therapeutics (for review, see Bray and Tartaglia, Medicinal Strategies in the Treatment of Obesity, Nature 404: 672-677 (2000)). Said modes of action include, but are not limited to: Melanocortin 4-receptor (MC4) agonists, melanin-concentrating hormone (MCH) antagonists, cannabinoid (CB1) antagonists or inverse agonists, serotonin and noradrenalin reuptake inhibitors, monoamine oxidase (MAO) inhibitors, neuropeptide Y (NPY) Y1 or Y5 antagonists, leptin analogues, leptin-receptor (Ob) agonists, noradrenergic α₁-receptor agonists, β₂-receptor agonists, 5-HT_{2C}- receptor agonists, dopamine D1 receptor agonists, histamine H3-receptor antagonists, corticotropin-releasing hormone (CRH)/urocortin receptor agonists, galanin receptor antagonists, orexin receptor antagonists, opiod mu and kappa receptor antagonists, cocaine- and amphetamine-regulated transcript (CART) receptor agonists, ApoA-IV receptor agonists, and amylin receptor agonists.

Acceptable modes of action for selecting compounds to be screened by the methods of the present invention do not include those compounds that stimulate the release of serotonin.

In addition to monitoring plasma AGRP level during clinical trials to determine the efficacy of an appetite suppressant drug in clinical development, AGRP level may be measured in a subject who is not taking part in a clinical trial to measure the progress of a therapeutic regime designed to alleviate obesity. Said therapeutic regime may consist of administering to the subject a single appetite suppressant or a combination of more than one appetite suppressant. Appetite suppressants may be pharmaceutical compositions in development and not yet marketed or pharmaceutical compositions already approved for the treatment of obesity. Additionally, the therapeutic regime may include other treatments such as diet and exercise.

To this end, the present invention relates to a method for following the progress of a therapeutic regime designed to alleviate obesity, comprising: (a) assaying a plasma sample from a subject to determine a level of AGRP at a first time point; (b) assaying a second plasma sample from the subject to determine a level of AGRP at a second time. point, wherein the therapeutic regime is followed by the subject between the first time point and the second time point; and (c) comparing said level at said second time point to the level determined in (a) as a determination of effect of said therapeutic regime.

Pharmaceutical compositions that may serve as appetite suppressants to be tested by the methods of the present invention may be administered to the subject by any of several modes of delivery known in the art. For example, the pharmaceutical formulations for use in the novel methods of screening of the present invention may be administered topically, subcutaneously, intramuscularly, orally, systemically and parenterally.

Pharmaceutically useful compositions to be screened by the methods of the present invention may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, or modified protein to be tested by the methods of the present invention.

Therapeutic or diagnostic compositions to be screened by the methods of the present invention are administered to an individual in amounts sufficient to treat disorders such as obesity. The effective amount may vary according to a variety of factors such as the individual's condition, weight, sex and age. Other factors include the mode of administration and the use of chemical derivatives.

The term “chemical derivative” describes a molecule that contains additional chemical moieties which are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Compounds to be screened according to the methods disclosed herein may be used alone at appropriate dosages. Alternatively, co-administration or sequential administration of other agents may be desirable.

The compositions to be tested according to this invention can be administered in a wide variety of therapeutic dosage forms in conventional vehicles for administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts.

Advantageously, compounds to be tested by the methods of the present invention may be administered to the subject in a single daily dose, or the total daily dosage may be administered in divided doses of two, three or four times daily. Furthermore, compounds to be tested by the methods of the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

For a therapeutic regime including combination treatment with more than one active agent, where the active agents are in separate dosage formulations, the active agents can be administered concurrently, or they each can be administered at separately staggered times.

All publications mentioned herein are incorporated by reference for the purpose of describing and disclosing the methodologies and materials that are disclosed therein that might be used in connection with the present invention. Nothing herein is to be construed as an admission that the invention is not entitled to antedate such disclosure by virtue of prior invention.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### EXAMPLE 1

### Plasma AGRP And Leptin Levels In Untreated Humans

A. Plasma AGRP Levels After Food Intake

Seventeen healthy human volunteers, ten males and seven females, were recruited. None of the study subjects had been diagnosed with diabetes mellitus. Study participants did not take any medication during the course of the study. Body weight and height were measured at study enrollment.

Study subjects did not ingest any food or drink overnight beginning twelve a.m. The next morning, a blood sample was taken from each volunteer at nine a.m. Immediately after the first blood sampling, study participants consumed a Western style breakfast of their choice (including milk, fruit juice, coffee, fruit salad, egg, bagel, muffin, cereal, and bread). Two hours later, a second blood sample was taken from each volunteer. Human blood samples were treated with EDTA. Plasma was collected and stored at −80° C.

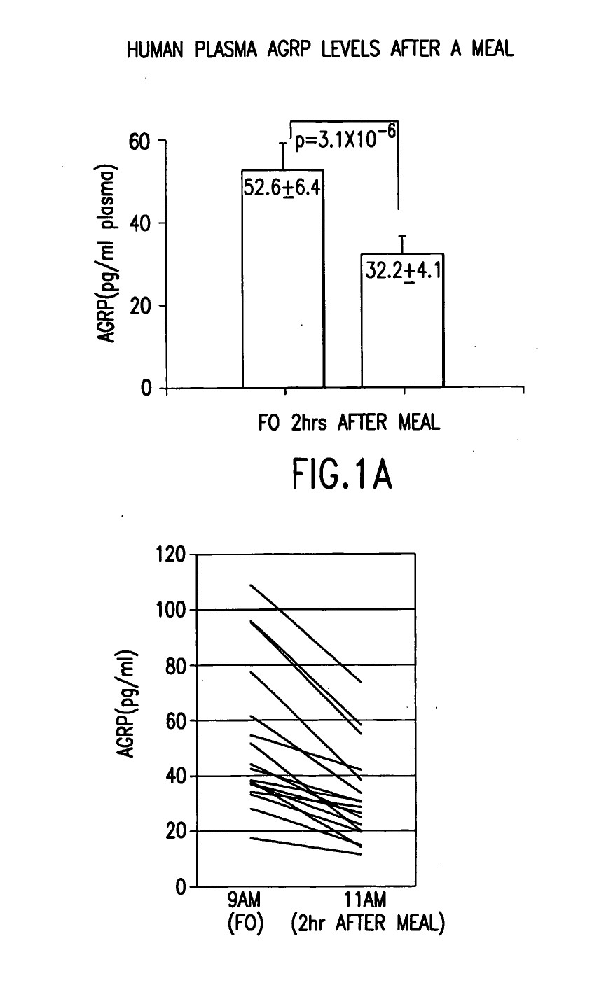

Plasma AGRP levels from blood taken at nine a.m. measured 52.6±6.4 pg/mL (mean±SE). The average plasma AGRP level decreased by 39% following a Western style breakfast (p=3.1×10⁻⁶) (see FIG. 1A).

B. Plasma AGRP Levels During Fasting

A follow-up study was conducted two months later in which fifteen of the original seventeen volunteers participated (eight males and seven females). The same procedure was followed as described above, except fasting continued following the nine a.m. blood sampling.

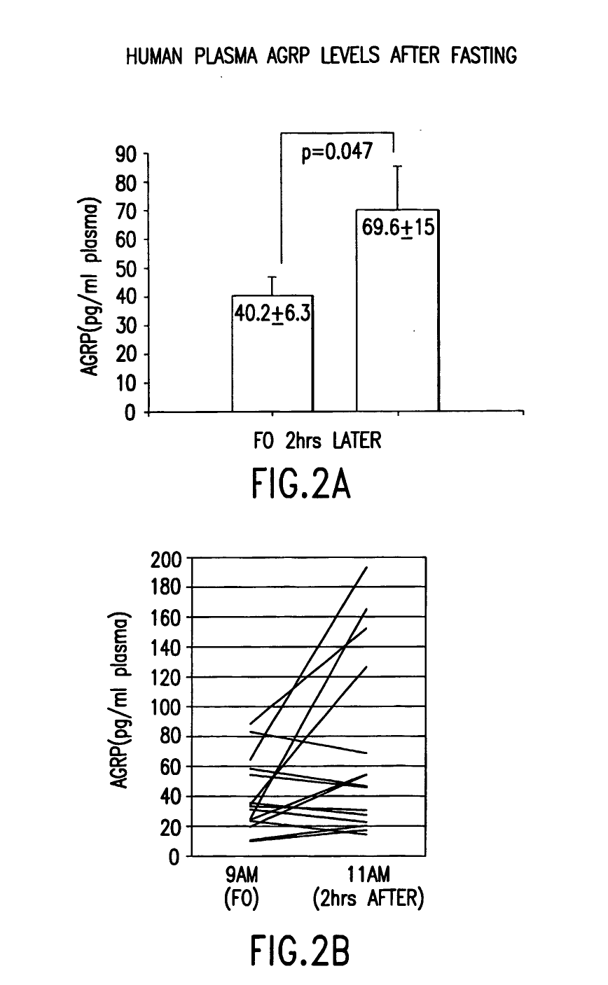

In this study, the initial average plasma AGRP level measured 40.2±6.3 pg/mL after an overnight fast. Two hours later, mean plasma AGRP levels in volunteers who continued to fast for an additional two hours had increased by 73% (FIG. 2A; p=0.047, paired t-test). More variability among plasma AGRP levels of individual subjects was observed in this study-arm compared to the previous study, in which plasma AGRP was determined after a meal. Plasma AGRP levels decreased in seven subjects, but increased in eight subjects after an additional two hours of fasting relative to levels obtained at the 9 a.m. blood sampling (see FIG. 2B). The data indicates that plasma AGRP levels decreased as a result of food intake in all subjects (see FIG. 1B), but that plasma AGRP levels increased as a result of an additional two hours of fasting compared to initial overnight fasting levels in only some subjects.

In addition to measuring plasma AGRP levels, the body mass index (BMI) of each of the original 17 study subjects was determined using the following formula: BMI=body weight in kg/(body height in m)². The results indicate that AGRP levels did not correlate with BMI (data not shown).

C. Plasma Leptin And Insulin Levels Of Human Volunteers

Both the hormone insulin and leptin, a peptide produced mainly by adipose cells, have been implicated in the central nervous system regulation of body weight and energy expenditure. (for review, see Schwartz et al., Nature 404: 661-671 (2000)). Signaling by leptin in the hypothalamic arcuate nucleus has been associated with reduced secretion of neuropeptide Y (NPY), reduced expression of AGRP, and increased expression of the α-melanocyte-stimulating hormone (α-MSH) precursor.

Ebihara and colleagues (Diabetes 48: 2028-2033 (1999)) have demonstrated that AGRP MRNA expression was increased in the hypothalamus of leptin-deficient ob/ob mice and leptin receptor-deficient db/db mice. This evidence, along with additional experiments from this group and others, suggests that AGRP can negatively regulate leptin signaling and that leptin can downregulate AGRP expression in the hypothalamus.

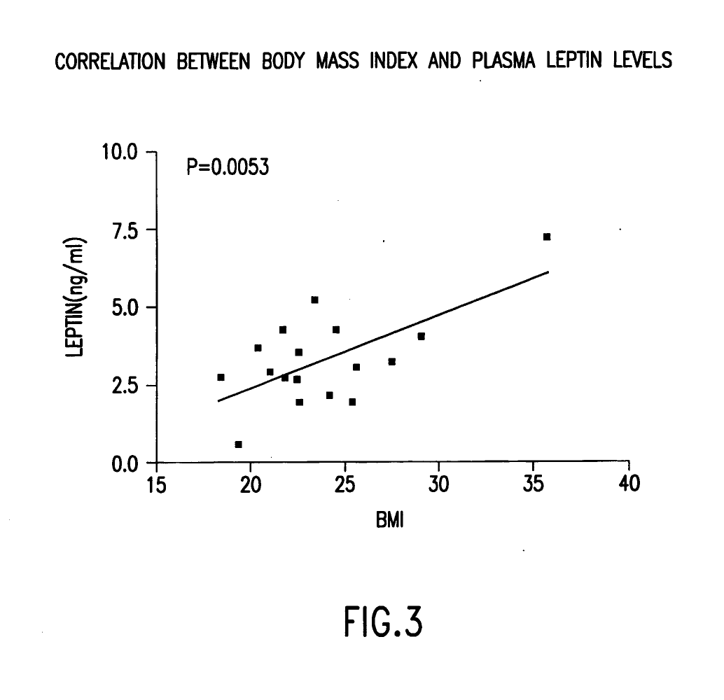

To determine if leptin levels were also correlated with food intake, and, therefore, whether plasma leptin was a candidate biomarker for appetite suppressant efficacy, we studied plasma leptin levels in each volunteer. Plasma leptin levels of each of the original 17 volunteers was measured after the overnight fasting period with a leptin RIA kit (Linco Research, Inc., St. Charles, Mo.) A significant difference in fasted versus fed plasma leptin levels could not be detected (data not shown). Plasma leptin levels correlated with BMI by linear regression (p=0.0053; FIG. 3), consistent with a previous study by Considine and colleagues (N. Engl. J. Med. 334: 292-295 (1996)) demonstrating that plasma leptin levels are elevated in obese human subjects relative to lean individuals.

Plasma insulin levels of the original 17 volunteers were also measured at 9 a.m. after the overnight fast with a human specific insulin RIA kit (Linco Research, Inc., St. Charles, Mo.). The average fasting insulin level was 7.7±0.57 μU/ml, indicating that the study subjects were not diabetic.

### EXAMPLE 2

### Plasma AGRP Level And Food Intake In Untreated DIO Rats

Three month-old, diet-induced obese (DIO) male Sprague-Dawley rats were obtained from Charles River Laboratories (Wilmington, Mass.). Rats were individually or group housed in a centralized vivarium and were exposed to a 12 h light,12 h dark cycle (lights on at 0400 h EST (LD)). The DIO rats were maintained on a medium high fat diet (D12266B, Research Diets, New Brunswick, N.J.) with ad libitum access to water. Treatment groups each consisted of six rats either fed ad libitum (blood collected at eight a.m.), fasted for forty eight hours (blood collected at eight a.m.), or fasted for forty eight hours and re-fed diet for two hours (blood collected at ten a.m.). Rats were euthanized by CO₂ inhalation and decapitated. Trunk blood was then collected from the rats and treated with EDTA. Plasma was collected and stored at −80° C. All animal protocols used in these studies were approved by the Merck Research Laboratories Institutional Animal Care and Use Committee in Rahway, N.J.

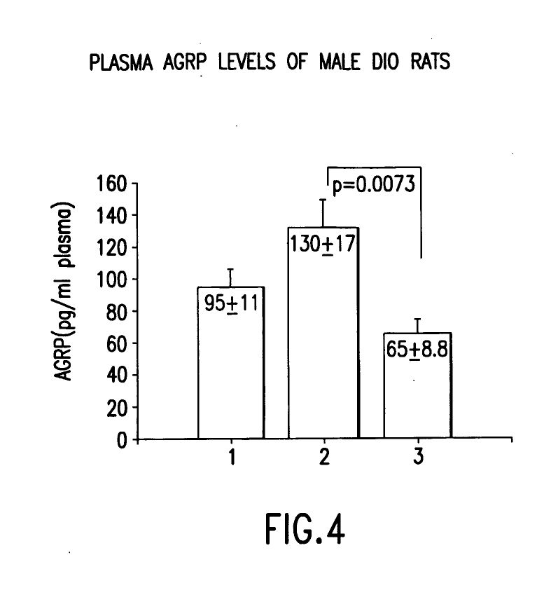

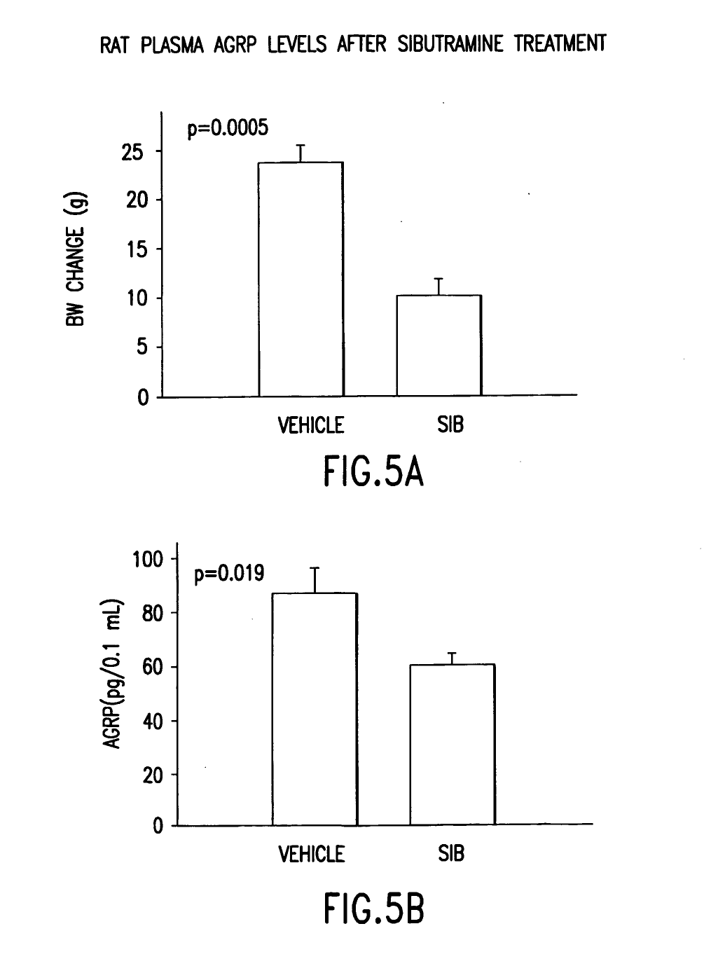

Plasma AGRP levels were low in the group that received ad libitum. food following a 48 hour fast (65±8.8 pg/ml), while the 48 hour fasted group showed the highest plasma AGRP levels (130±17 pg/ml; p<0.01 compared to feeding following a fast; FIG. 4). The ad libitum fed group had intermediate plasma AGRP values (95±11 pg/ml; not statistically significantly different from either group). These data are consistent with an effect of food intake on plasma AGRP levels in rats.

### EXAMPLE 3

### Plasma Analysis

A human AGRP RIA assay kit (Phoenix Pharmaceuticals, Inc., Belmont, Calif.), a human leptin RIA kit (Linco Research, Inc., St. Charles, Mo.), and a human specific insulin RIA kit (Linco Research, Inc., St. Charles, Mo.), were used to measure AGRP, leptin, and insulin, respectively in human plasma samples. A Phoenix AGRP RIA kit, which contains synthetic human AGRP (aa83-132) as a standard, was also used to measure AGRP level in rat plasma samples. The assay showed no significant cross-reactivity with leptin, orexin A, orexin B, neuropeptide Y, α-MSH, melanin-concentration hormone, and calcitonin gene related peptide. For the AGRP RIA, 1 mL human plasma or 100 μl rat plasma was used per assay, which are capable of detecting AGRP at levels from 1 to 128 pgs. Peptides were extracted from each plasma sample by 60% acetonitrile (HPLC Grade) in 1% trifluoroacetic acid followed by separation through a C18 columns, following the suggested procedures of the manufacturer. Statistical comparison is based on one-way ANOVA, t or paired t test.

### EXAMPLE 4

### Plasma AGRP Level In Lean Rats After Sibutramine Treatment

To determine if plasma AGRP levels of a subject are affected by the subject's intake of appetite suppressants, we conducted a four day dosing experiment in which the tertiary amine, N-{1-[1-(4-chlorophenyl)cyclobutyl]-3-methylbutyl}-N,N-dimethylamine hydrochloride monohydrate (hereinafter sibutramine) was administered to lean rats. Sibutramine is a known appetite suppressant which exerts its therapeutic effect by inhibiting noradrenalin and serotonin (5HT) reuptake in the central nervous system. Evidence indicates that sibutramine contributes to weight loss by both enhancing satiety and by increasing thermogenesis (for review, see Heal et al., Int. J. Obesity 22, Suppl 1: S18-S28 (1998)).

Male Sprague-Dawley rats (Charles River Laboratories, Wilmington, Mass.), which were 71 days of age at the beginning of the experiment, were treated with either vehicle alone (0.5% methyl cellulose; n=5 rats) or sibutramine (3 mg/kg of body weight in 0.5% methyl cellulose; n=7 rats). The treatment regime consisted of a single daily oral dose of the test compound every day for 4 days.

The body weight of each rat was measured before commencement of the experiment and at its conclusion. After 4 days of treatment with either test compound, plasma samples of each rat were collected for the measurement of AGRP level. An AGRP RIA assay kit (Phoenix Pharmaceuticals, Inc., Belmont, Calif.) was used to measure AGRP, which detects AGRP at levels from 1 to 128 pg. The AGRP kit contains a synthetic human AGRP fragment (aa83-132) as the standard. The assay showed no significant cross-reactivity with leptin, Orexin A, Orexin B, neuropeptide Y, α-MSH, melanin-concentration hormone, and calcitonin gene related peptide. For AGRP RIA, 100 μl rat plasma was used per assay. Peptides were extracted from each plasma sample by 60% acetonitrile (HPLC Grade) in 1% trifluoroacetic acid followed by separation through a conventional C18 column, following the manufacturer's suggested procedures. Statistical comparison is based on unpaired t test.

After 4-days of treatment, the vehicle group gained 23.6±4.16 g while the sibutramine-treated group gained 10±4.76 g (mean±standard error). The difference in body weight gain was statistically significant (p=0.0005, FIG. 1). Plasma AGRP level of the vehicle and Sibutramine treated group was 86.6 +21.8 and 60.1±10.9 pg/100 μplasma respectively. The difference in plasma AGRP level between rats treated with sibutramine and rats treated with vehicle alone was also statistically significant (p=0.019, FIG. 2).

### EXAMPLE 5

### Plasma AGRP Level In Lean Rats after MC4R Agonist Treatment

The involvement of the melanocortin system and its regulation of body weight has been intensely studied due to the prevalence of genetic defects affecting this system in the mouse. Additionally, mutations in pro-opiomelanocortin (POMC), the α-melanocyte stimulating hormone (α-MSH) precursor, and melanocortin 4 receptor (MC4R) have been reported in obese humans. Mice lacking the MC4 receptor become extremely obese, suggesting that agonists of this receptor are a therapeutic target for obesity.

To determine if plasma AGRP level correlates with a subject's intake of MC4R agonists given as appetite suppressants, we measured plasma AGRP level in DIO rats after treatment with specific MC4 agonists. Male DIO rats were treated either with an MC4 agonist (15 mg/kg, po, b.i.d., for 4 days, n=7) or with vehicle (n=7). The specific MC4 agonist used for treatment was (aR)-N,N-bis(4-[(tert-butylamino)carbonyl]-4-cyclohexyl 1-{4-fluoro-N-[(2-methyl-2-azabicyclo[2.2.1]hept-6-yl)carbonyl]-D-phenylalanyl}piperidin-2-yl)-4-fluoro-N-[(2-methyl-2-azabicyclo[2.2.1]hept-6-yl)carbonyl]-D-phenylalaninamide hydrochloride (hereinafter Compound A). Compound A is a bridged piperidine derivative which was shown to be a selective MC4 agonist, and, therefore, useful for the treatment of obesity.

Body weight of each rat was measured at the beginning and end of the experiment. After 4 days of treatment with Compound A or with vehicle, plasma samples were obtained from each rat. Plasma AGRP level was thereafter determined using an RIA Kit from Phoenix Pharmaceuticals, Inc. (Belmont, Calif.).

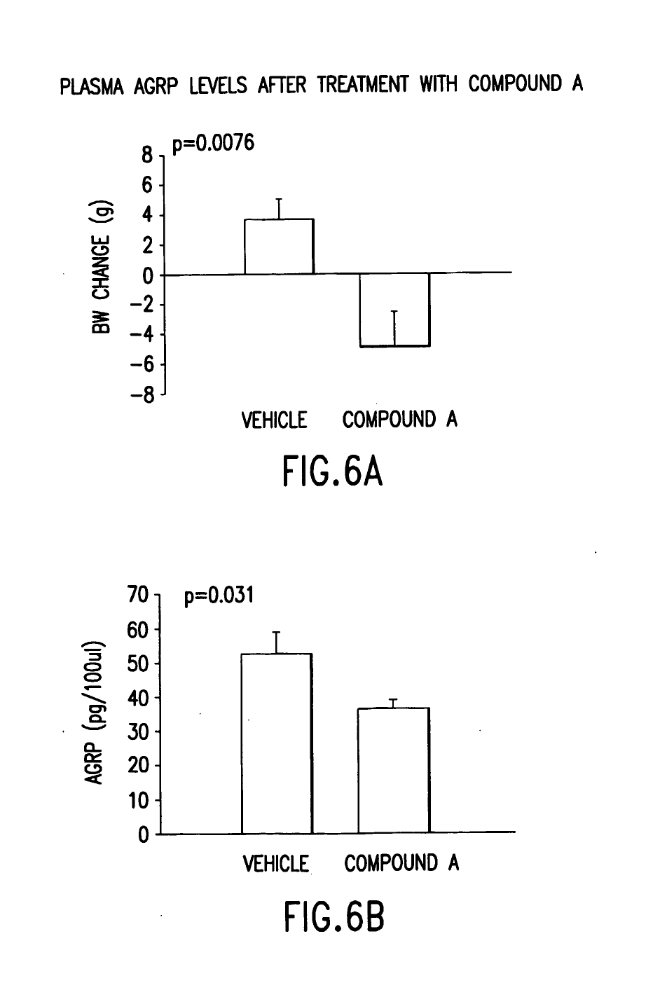

After 4-days of treatment, the vehicle group gained 3.7±1.4 g of body weight while the Compound A group lost 4.9±2.3 g of body weight (mean±standard error, TABLE 1). The difference in body weight between rats treated with Compound A and rats treated with vehicle was statistically significant (p=0.0076, FIG. 6A). Plasma AGRP levels of the vehicle and Compound A treated group were 5.25±6.2 and 36.2±2.5 pg/100 μl plasma respectively (TABLE 1). The difference in plasma AGRP level between rats treated with Compound A and rats treated with vehicle was also statistically significant (p=0.031, FIG. 6B). Statistical analyses were carried out using unpaired t-tests.

TABLE 1

| | Vehicle | | Compound A | |
|---|---|---|---|---|
| | BW change | AGRP | BW change | AGRP |
| | (g) | (pg/0.1 mL) | (g) | (pg/0.1 mL) |
| 1 | 7 | 68.6 | −5 | 46.2 |
| 2 | 8 | 40.9 | −7 | 33 |
| 3 | 2 | 74.7 | −7 | 26.5 |
| 4 | 1 | 57.9 | −16 | 31.6 |
| 5 | 6 | 55.4 | 2 | 35.8 |
| 6 | −2 | 30.5 | 1 | 42.2 |
| 7 | 4 | 39 | −2 | 38.1 |
| Mean | 3.7 | 52.5 | −4.9 | 36.2 |
| Std Error | 1.4 | 6.2 | 2.3 | 2.5 |

In a different experiment, groups of male DIO rats were treated either with a second MC4R agonist (20 mg/kg, po, b.i.d., for 4 days, n=6) or with vehicle (n=5). In this study, the specific MC4 agonist used was 4-[2-methylaminocarbonyl-4-flurorphenyl]-1-{[(3S, 4R)-1-tert-butyl-4-(2,4-difluorophenyl)-pyrrolidin-3-yl]carbonyl}piperidine (hereinafter Compound B). As above, body weight of each rat was measured at the beginning and end of the experiment. For each treatment group, plasma samples were collected after 4 days of treatment and AGRP level was measured using an RIA Kit from Phoenix Pharmaceuticals, Inc. (Belmont, Calif.).

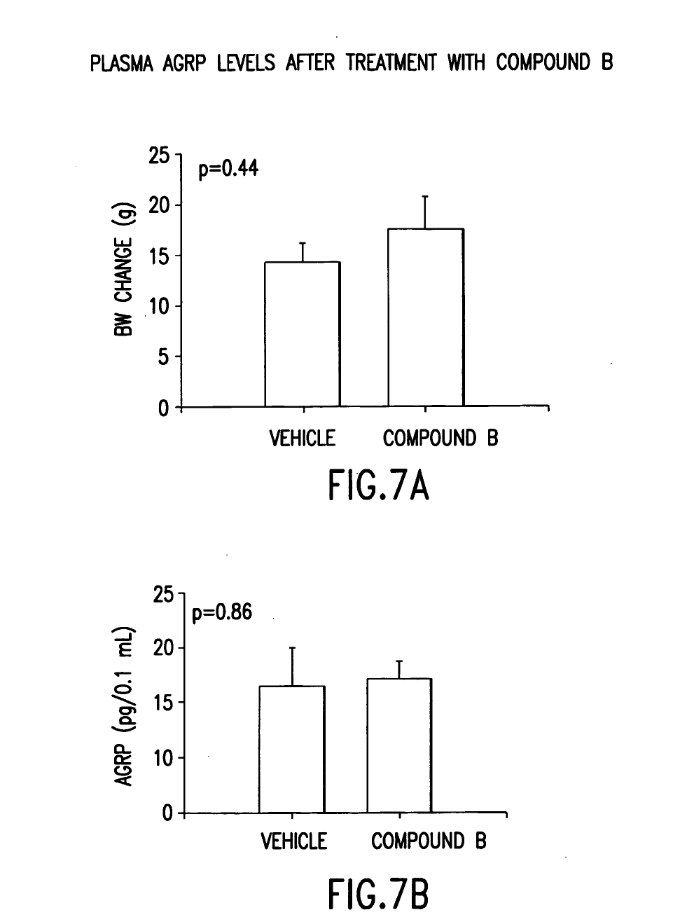

After 4-days of treatment, the vehicle group gained 14.2±1.9 g body weight and the Compound B-treated group gained 17.5±3.3 g of body weight (mean ±standard error, TABLE 2). The difference in body weight between rats treated with Compound B and rats treated with vehicle was not statistically significant (p=0.44, unpaired t-test, FIG. 7A).

Plasma AGRP levels of the vehicle and Compound B treated groups were 45.8±14 and 48.4±6.3 pg/100 μl plasma, respectively (TABLE 2). The difference in plasma AGRP level between rats treated with compound B and rats treated with vehicle alone was not statistically significant (p=0.86, unpaired t-test, FIG. 7B). This result is consistent with the above data showing that Compound B was not effective at reducing body weight in rats. While not wishing to be bound by theory, it is possible that Compound B, which, like Compound A, is a selective MC4R agonist, was unable to reduce body weight in DIO rats because it is less potent than Compound A, less bioavailable than Compound A or less brain penetrable than Compound A.

TABLE 2

| | Vehicle | | Compound B | |
|---|---|---|---|---|
| | BW change | AGRP | BW change | AGRP |
| | (g) | (pg/0.1 mL) | (g) | (pg/0.1 mL) |
| 1 | 16 | 58.8 | 23 | 39.8 |
| 2 | 11 | 23.3 | 25 | 66.9 |
| 3 | 9 | 14.6 | 15 | 47 |
| 4 | 15 | 92.8 | 12 | 64.8 |
| 5 | 20 | 39.5 | 25 | 45.6 |
| 6 | | | 5 | 26 |
| mean | 14.2 | 45.8 | 17.5 | 48.4 |
| Std Error | 1.9 | 14 | 3.3 | 6.3 |

### EXAMPLE 6

### Plasma AGRP Level In Lean Rats After S(+) Fenfluramine Treatment

The serotonin pathway has been implicated in the regulation of food intake and body weight control. Evidence indicates that serotonin receptors have a role in regulating both the quantity of food intake and macronutrient selection. To determine if plasma AGRP level was affected by a subject's intake of appetite suppressants that stimulate release of serotonin (5HT), we measured plasma AGRP level in lean rats treated with S(+) fenfluramine (interchangeably used herein with dexfenfluramine), an enantiomer of fenfluramine. Dexfenfluramine was approved by the FDA in 1994 for the long-term treatment of obesity. The therapeutic effect of fenfluramine likely occurs through 5-HT_{2C} receptors, since fenfluramine-induced reduction in fat intake is diminished in 5-HT_{2C} knockout mice (Vickers et al., Psychopharmacology 143: 309-314 (1999)). Both fenfluramine and dexfenfluramine were removed from the market due to a later-discovered association with valvulopathy.

Male DIO rats were treated either with S(+) fenflurarnine (3 mg/kg, po, b.i.d., for 4 days, n=7) or with vehicle (n=5). Body weight of each rat was measured at the beginning and end of the experiment. After 4 days of treatment with Compound A or with vehicle, plasma samples were obtained from each rat. Plasma AGRP level was thereafter determined using an RIA Kit from Phoenix Pharmaceuticals, Inc. (Belmont, Calif.).

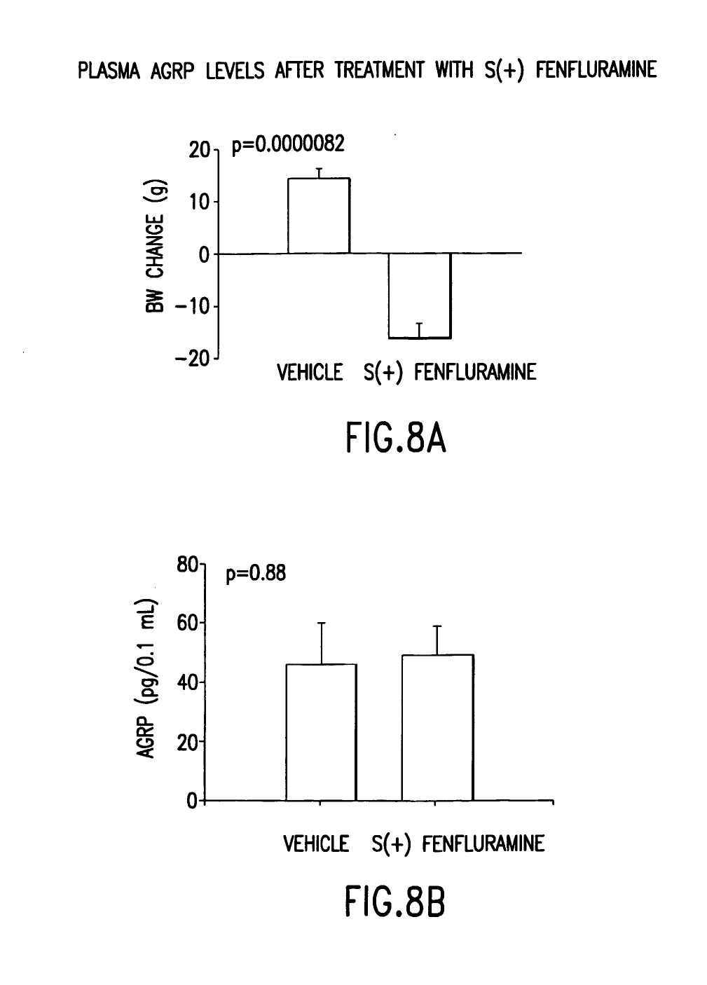

After 4-days of treatment, the vehicle group gained 14.2±1.9 g of body weight and the S(+) fenfluramine-treated group lost 16.3±2.7 g of body weight (mean±standard error, TABLE 3). The difference in body weight change was statistically significant (p=0.0000082, unpaired t-test, FIG. 8). The average plasma AGRP levels of the vehicle and S(+) fenfluramine treated groups were 45.8±14 and 48.3±9.9 pg/100 μl plasma, respectively (TABLE 3). Despite the statistically significant difference in body weight between the vehicle and fenfluramine-treated groups, the difference in plasma AGRP levels was not statistically significant (p=0.88, FIG. 8). While not wishing to be bound by theory, it is possible that the serotonin releasing effect of fenfluramine is causing a compensatory response masking the change of plasma AGRP level.

TABLE 3

| | Vehicle | | S(+) fenfluramine | |
|---|---|---|---|---|
| | BW change | AGRP | BW change | AGRP |
| | (g) | (pg/0.1 mL) | (g) | (pg/0.1 mL) |
| 1 | 16 | 58.8 | −10 | 62.6 |
| 2 | 11 | 23.3 | −15 | 94.4 |
| 3 | 9 | 14.6 | −28 | 16.9 |
| 4 | 15 | 92.8 | −10 | 44.6 |
| 5 | 20 | 39.5 | −25 | 39.3 |
| 6 | | | −13 | 22.8 |
| 7 | | | −13 | 57.8 |
| mean | 14.2 | 45.8 | −16.3 | 48.3 |
| Std Error | 1.9 | 14 | 2.7 | 9.9 |

### EXAMPLE 7

### Plasma AGRP Level In Lean Rats After Treatment With AM251, A Cannabinoid Inverse Agonist

Evidence suggests that cannabinoid receptors have a role in controlling appetite and body weight. Intake of Δ⁹-tetrahydrocannabinol has been described as an appetite stimulant. Cannabinoids were also reported to increase feeding in animals. Colombo and colleagues (Pharmacol. Lett. 63(8):113-117 (1998)) have reported a reduction of food intake and body weight in lean and obese rats after administration of a CB1 receptor antagonist.

To determine if plasma AGRP levels of a subject are affected by the subject's intake of an appetite suppressant that exerts its effect through the CB1 receptor, a human CB1 receptor inverse agonist was administered to lean rats.

AM251 (N-(piperidin-1-yl)-5-(4-iodophenyl)-1(2,4-dichlorophenyl)4-1H-pyrazole-3-carboxamide; Tocris Cookson Inc., Ellisville, Mo.), a human CB1R inverse agonist, was tested for its effects on body weight and plasma AGRP level is a 5-day dosing experiment in male Sprague-Dawley rats (Charles River ories, Wilmington, Mass.). Rats were 15 weeks of age at the beginning of the experiment. Groups of rats were treated with either vehicle alone (5% Tween 80/0.5% methyl cellulose; 7 rats) or AM251, a CB1R inverse agonist (6 rats, see Gatley et al., Eur. J. Pharmacol. 307: 331-38 (1996)). For AM251-treated rats, the treatment regime consisted of a single daily dose of AM251 at 10 mpk orally every day for 5 days. Body weight of each rat was measured at the beginning and the end of the experiment.

Two hours after the last dose of AM251 or vehicle alone was given, plasma samples of each rat were collected for the measurement of plasma AGRP level. An AGRP RIA assay kit (Phoenix Pharmaceuticals, Inc., Belmont, Calif.), which detects AGRP at levels from 1 to 128 pg ,was used to measure AGRP levels. The assay showed no significant cross-reactivity with leptin, Orexin A, Orexin B, neuropeptide Y, α-MSH, melanin-concentration hormone, or calcitonin gene related peptide. For AGRP RIA, 100 μl rat plasma was used per assay. Peptides were extracted from each plasma sample by 60% acetonitrile (HPLC Grade) in 1% trifluoroacetic acid followed by separation through a conventional C18 column, as suggested by the manufacturer. Statistical comparison was based on an unpaired t test.

After five days of treatment, the vehicle group gained 7.00±3.82 g of body weight while the AM251 treated group lost 12.8±1.22 g (mean±standard error). The difference in body weight change was statistically significant (p=0.00076, FIG. 9A), suggesting that the CB1R inverse agonist is an effective compound for the treatment of obesity. Plasma AGRP levels of the vehicle and AM251-treated groups were 26.6±4.28 and 9.56±1.43 pg/100 μl plasma, respectively. The difference in plasma AGRP levels was also statistically significant (p=0.0048, FIG. 9B). The correlation between plasma AGRP decrease and body weight change after AM251 treatment supports the notion that plasma AGRP level may serve as a biomarker of satiety.

1. A method of determining the efficacy of a test compound given to a subject for the treatment of obesity, comprising:
(a) assaying a plasma sample from the subject to determine a level of agouti related protein (AGRP) at a first time point;
(b) administering the test compound to the subject; and
(c) thereafter assaying a plasma sample from the subject to determine the level of AGRP at a second time point;
wherein the test compound is an appetite suppressant which does not stimulate the release of serotonin and wherein a decreased level of AGRP at the second time point relative to the first time point is indicative of the efficacy of the test compound in treating obesity.

2. The method of claim 1, wherein the subject is a human.

3. The method of claim 1, wherein the subject is a rodent.

4. The method of claim 3, wherein the rodent is a rat.

5. The method of claim 1, wherein the level of AGRP is determined by radioimmunoassay.

6. The method of claim 1, wherein the level of AGRP is determined by ELISA.

7. The method of claim 1, wherein the level of AGRP is determined by radioligand binding assay.

8. The method of claim 1, wherein the level of AGRP is determined by liquid chromatography.

9. The method of claim 1, wherein the amount of time between the first time point and the second time point is at least four hours.

10. The method of claim 1, wherein the amount of time between the first time point and the second time point is from about two hours to about four days.

11. A method for following the progress of a therapeutic regime designed to alleviate obesity, comprising:
(a) assaying a plasma sample from a subject to determine a level of agouti related protein (AGRP) at a first time point;
(b) assaying a second plasma sample from the subject to determine a level of AGRP at a second time point, wherein the therapeutic regime is followed by the subject between the first time point and the second time point; and
(c) comparing said level at said second time point to the level determined in (a) as a determination of effect of said therapeutic regime.

12. A method for determining the appropriate dosage of an appetite suppressant given to a subject for the treatment of obesity, comprising:
(a) assaying a plasma sample from the subject to determine a level of agouti related protein (AGRP) at a first time point;
(b) administering the appetite suppressant to the subject;
(c) thereafter assaying a plasma sample from the subject to determine the level of AGRP at a second time point, wherein the appetite suppressant does not stimulate the release of serotonin;
(d) determining whether the appetite suppressant was administered at the appropriate dosage, wherein a decreased level of AGRP at the second time point relative to the first time point is indicative of the efficacy of the appetite suppressant in treating obesity at the dosage administered; and
(e) adjusting dosage as needed.
